(19) United States
(12) Plant Patent Application Publication
(10) Pub. No.: US 2003/0208810 P1
(43) Pub. Date: Nov. 6, 2003

(54) CHRYSANTHEMUM PLANT NAMED 'GEDI TWO LUC'

(76) Inventor: Dirk Pieters, Staden (BE)

Correspondence Address: KNOBBE MARTENS OLSON & BEAR LLP, 2040 MAIN STREET, FOURTEENTH FLOOR, IRVINE, CA, 92614, US

(21) Appl. No.: 10/137,809

(22) Filed: May 1, 2002

(51) Int. Cl. A01H 5/00

(52) U.S. Cl. PLT/287

(57) ABSTRACT

A new and distinct Chrysanthemum plant cultivar is disclosed, characterized by a decorative-type inflorescence, consistent flowering response to short days, blooming consistently after 49 days of short day length, free branching habit, Gray-orange ray florets that darken as the inflorescence ages, a very uniform round growth habit, and a large quantity of blooms per flowering branch.

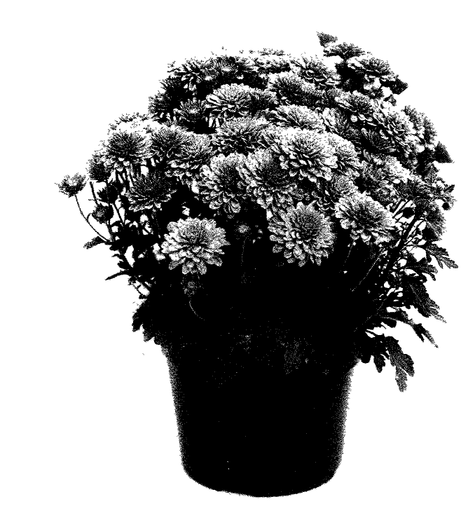

### LATIN NAME OF THE GENUS AND SPECIES

The present invention relates to a new and distinct cultivar of Chrysanthemum plant, botanically known as Dendranthema morifolium.

### VARIETY DENOMINATION

The new and distinct Chrysanthemum plant is hereinafter referred to by the cultivar name ‘GEDI TWO LUC.’

### BACKGROUND OF THE INVENTION

The new cultivar is a product of hybridization of a female parent Chrysanthemum plant variety ‘GEDI VL8’ (U.S. Plant Pat. No. 11,344) and male parent Chrysanthemum plant variety ‘Sapiro Brons’ (undistributed in the United States). The new cultivar was discovered and selected by the inventor in October 1998.

Asexual reproduction of the new cultivar by apical tip cutting and meristem tissue culture was performed in Oxnard, Calif. and has shown that the unique features of this cultivar are stable and reproduced true to type on successive generations.

### SUMMARY OF THE INVENTION

The cultivar ‘GEDI TWO LUC’ has not been observed under all possible environmental conditions. The phenotype may vary somewhat with variations in environment such as temperature, daylength, and light intensity, without, however, any variance in genotype.

The following traits have been repeatedly observed and are determined to be the unique characteristics of ‘GEDI TWO LUC.’ These characteristics in combination distinguish ‘GEDI TWO LUC’ as a new and distinct Chrysanthemum cultivar:

1. Decorative-type inflorescence,

2. Consistent flowering response to short days, blooming consistently after 49 days of short day length,

3. Free branching habit,

4. Gray-orange ray florets that darken as the inflorescence ages,

5. Very uniform round growth habit, and

6. Large quantity of blooms per flowering branch.

Plants of the new cultivar are similar to plants of the female parent variety, ‘GEDI VL8’ in most horticultural characteristics, however plants of the new cultivar have more blooms per flowering branch, smaller diameter inflorescences, and naturally bloom two weeks earlier than plants of the female parent variety.

Plants of the new cultivar are similar to plants of the male parent variety, ‘Sapiro Brons’ in most horticultural characteristics, however plants of the new cultivar have a wider diameter inflorescence, more ray florets per inflorescence, and naturally bloom three weeks earlier than plants of the male parent variety.

In comparison to the commercially available variety ‘GEDI ORO8’ (U.S. Plant Pat. No. 11,569), ‘GEDI TWO LUC’ blooms two weeks earlier and has more blooms per flowering branch and many more ray florets per inflorescence.

### BRIEF DESCRIPTION OF THE PHOTOGRAPH

The photograph shows a plant of ‘GEDI TWO LUC’ grown in a six-inch container. One cutting was used in the pot.

### DETAILED BOTANICAL DESCRIPTION

In the following description, color references are made to The Royal Horticultural Society Colour Chart except where general terms of ordinary dictionary significance are used. The following observations and measurements describe plants grown in Oxnard, Calif. during the month of December and January. The growing temperature ranged from 55° to 68° F. at night to 60° to 75° F. during the day. Measurements and numerical values represent averages of typical flowering types.

Botanical classification: Dendranthema morifolium cultivar ‘GEDI TWO LUC’

Commercial classification: Garden-type Chrysanthemum

Parentage: Hybridization of a female parent Chrysanthemum plant variety, ‘GEDI VL8’ (U.S. Plant Pat. No. 11,344), and a male parent Chrysanthemum plant variety ‘Sapiro Brons’ (undistributed in the United States)

### PROPAGATION

Time to Rooting: 7 to 14 days at approximately 21° C.

Rooting habit: Fine, fibrous

### PLANT

Growth Habit: Mounding herbaceous perennial

Height: Approximately 20 cm

Spread: Approximately 18 cm

Growth rate: Moderate

Branching Characteristics: Free Branching

Length of Lateral Branches: Approximately 18 cm

Number of leaves per lateral branch: Approximately 48

### FOLIAGE

Leaf:

Arrangement.—Alternate.

Average Length.—Approximately 7.2 cm.

Average Width.—Approximately 3.5 cm.

Shape of blade.—Ovate.

Apex.—Cuspidate.

Base.—Attenuate.

Attachment.—Stalked.

Margin.—Palmately lobed.

Texture of top surface.—Lightly pubescent.

Texture of bottom surface.—Lightly pubescent.

Color.—young foliage upper side: Near 137A young foliage under side: Near 137C mature foliage upper side: Near 137A mature foliage under side: Near 137C venation upper side: Near 138C venation underside: Near 138B.

Type.—Net.

Petiole:

Average Length.—Approximately 2.1 cm.

Color.—Near 138B.

Diameter.—Approximately 0.25 cm.

### BLOOM

Inflorescence:

Flowering habit.—induced by darkness period greater than 13.5 hours, approximately 49 days of appropriate day length required to induce and develop blooms.

Inflorescence form.—Decorative.

Natural flowering season.—Approximately the last two weeks of September.

Number of inflorescences per lateral branch.—Approximately 32.

Inflorescence diameter.—Approximately 4.5 cm.

Inflorescence depth.—Approximately 1.5 cm.

Inflorescence longevity on plant.—Approximately 28 days.

Persistence.—Persistent.

Ray Florets:

Appearance.—matte.

Texture.—smooth.

Average Number per flower.—203.

Shape.—Oblong.

Margin.—Entire.

Apex.—Retuse.

Length.—Approximately 1.6 cm.

Width.—Approximately 0.5 cm.

Color.—upper surface at first opening: outer ray florets: Near 167A inner ray florets: Near 1B upper surface at maturity: Near 163A upper surface at fading: Near 171B under surface at first opening: Near 164A under surface at maturity: Near 174C under surface at fading: Near 166C.

Disc florets:

Not present.

Peduncle:

Length.—at terminal end (shortest): Approximately 4.6 cm at lateral end (longest): Approximately 7.6 cm.

Angle to stem.—Acute.

Strength.—Moderate.

Color.—Near 138B.

Habit.—Upright.

Diameter.—Approximately 0.2 cm.

Inflorescence bud:

Length.—Approximately 1 cm.

Diameter.—Approximately 0.9 cm.

Form.—Globular.

Color.—Near 172B.

Involucral Bracts (Phyllaries):

Appearance.—Matte.

Texture.—Lightly pubescent.

Number.—Approximately 104.

Shape.—Oblanceolate.

Margin.—Entire.

Apex.—Acute.

Length.—Approximately 0.7 cm.

Width.—Approximately 0.2 cm.

Color.—upper side: Near 136A.

### REPRODUCTIVE ORGANS

Ray Florets.—

Pistillate.—Number of pistils per flower: 1 Stigma shape: 2 branched Stigma color: Near 7A Style color: Near 154D Style length: Approximately 0.4 cm.

What is claimed is:

1. A new and distinct cultivar of Chrysanthemum plant named ‘GEDI TWO LUC’ as herein illustrated and described.
